(19) United States
(12) Patent Application Publication
(10) Pub. No.: US 2005/0209131 A1
(43) Pub. Date: Sep. 22, 2005

(54) Composition containing peptides complexed with a copper ion

(76) Inventors: Laura C. Singleton, Los Angeles, CA (US); Lewis C. Bealer, Santa Monica, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/130,336

(22) Filed: May 16, 2005

Related U.S. Application Data
(63) Continuation of application No. 09/991,293, filed on Nov. 16, 2001, now abandoned.

(51) Int. Cl. A61K 38/08; A61K 7/11

(52) U.S. Cl. 514/6; 424/70.14

(57) ABSTRACT

The present invention relates to compositions comprising peptides complexed with a copper ion. In one embodiment, the composition comprises a copper peptide and a basic amino acid. In another embodiment, the composition is an emulsion composition comprising a copper peptide and a non-ionic emulsifier, wherein the composition is substantially free of ionic emulsifiers.

### FIELD OF THE INVENTION

The present invention relates to compositions comprising peptides complexed with a copper ion.

### BACKGROUND OF THE INVENTION

Over the past few years, a number of skin care products have entered the market place containing the peptides complexed with a copper ion (hereinafter “copper peptide”). E.g., Neova® Night Therapy, Neutrogena® Visibly Firms™ Night Cream, and Neutrogena® Visibly Firm™ Eye Cream. Copper peptide has been reported to be efficacious in wound healing, minimization of fine line and wrinkles, stimulation of dermal collagen, enhancement of skin elasticity, stimulating hair growth, and skin moisturization.

Many of these compositions, however, lack stability at elevated temperatures as (i) the copper ion was found to dissociate from the peptide and (ii) the peptide was found to degrade over time. This dissociataion and degredation both reduces efficacy as well as changes the color of the composition, thereby making it less consumer desirable. The present invention relates to a method of stabilizing copper peptide in a topical composition, thereby, creating a more efficacious and storage stable product.

### SUMMARY OF THE INVENTION

The invention features a composition comprising a peptide complexed with a copper ion (hereinafter a “copper peptide”). In one embodiment, the composition comprises a copper peptide and a basic amino acid. In another embodiment, the composition is an emulsion composition comprising a copper peptide and a non-ionic emulsifier, wherein the composition is substantially free of ionic emulsifiers. In one embodiment, the composition has a pH from about 6 to about 8 such as a pH of about 7.

Other features and advantages of the present invention will be apparent from the detailed description of the invention and from the claims

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. As used herein, all percentages are by weight unless otherwise specified.

### DEFINITIONS

As used herein, “topical application” means directly laying on or spreading on outer skin using, e.g., by use of the hands or an applicator such as a wipe.

As used herein, “cosmetically-acceptable” means that the copper peptide, cosmetically active agents or inert ingredients which the term describes are suitable for use in contact with tissues (e.g., the skin) without undue toxicity, incompatibility, instability, irritation, allergic response, and the like, commensurate with a reasonable benefit/risk ratio.

As used herein, “safe and effective amount” means an amount of compound or composition (e.g., the peptide complexed with a copper ion) sufficient to significantly induce a positive modification in the condition to be regulated or treated, but low enough to avoid serious side effects. The safe and effective amount of the compound or composition will vary with the particular condition being treated, the age and physical condition of the end user, the severity of the condition being treated/prevented, the duration of the treatment, the nature of concurrent therapy, the specific compound or composition employed, the particular cosmetically-acceptable topical carrier utilized, and like factors.

As used herein, “substantially free” means less than about 1%, by weight, preferably less than about 0.5%, and most preferably none.

Peptide Complexed with a Copper Ion

The compositions of the present invention comprise cooper peptides. What is meant by a “copper peptide” is a peptide complexed with a copper ion. Examples of such copper peptides are set forth in U.S. Pat. Nos. 4,665,054, 4,760,051, 4,810,693, 4,877,770, 5,135,913, 5,348,943, 5,382,431, and 5,550,183. In one embodiment, the peptide has from 3 to 10 amino acids. In one embodiment, the peptide is of the formula 1:
[>A1-A2-His-A3-A4-R3]ₙ:copper(II) R2 Formula 1
wherein A1 is Gly or absent; A2 is Gly, Lys, Ala, Ser, or Val; A3 is Lys or Gly; A4 is Trp, (Gly)ₙ-Trp where n is from 1 to 4, Pro-Val-Phe-Val, Val-Phe-Val, or absent; each R1 and R2, independently, is H, C₁₋₁₂ alkyl, C₇₋₁₀ phenylalkyl, or C(═O)E₁, where E₁ is C₁₋₂₀ alkyl, C₃₋₂₀ alkenyl, C₃₋₂₀ alkynyl, phenyl, 3,4-dihydroxyphenylalkyl, naphthyl, or C₇₋₁₀ phenylalkyl; provided that when either R1 or R2 is C(═O)E₁, the other must be H; R3 is OH, NH₂, C₁₋₁₂ alkoxy, C₇₋₁₀ phenylalkoxy, C₁₁₋₂₀ naphthylalkoxy, C₁₋₁₂ alkylamino, C₇₋₁₀ phenylalkylamino, or C₁₁₋₂₀ naphthylalkylamino; and n is 1 or 2. Copper (II) may be bound to one or more counter anions. Examples of additional counter anions include, but are not limited to, halides such as chloride, acetates, phosphonates, and sulfates, e.g., copper diacetate.

In one embodiment, A1 is absent. In one embodiment, A2 is Gly, Lys, or Ala. In one embodiment, A3 is Lys or Gly. In one embodiment, A4 is absent. In one embodiment, R1 and R2 are both H. In one embodiment, R3 is OH, NH2, or C₁₋₁₂ alkoxy.

In one embodiment, the peptide is [H₂-Gly-His-Lys-OH]ₙ:copper(II), [H₂-Gly-His-Lys-NH₂]ₙ:copper(II) (Copper Tripeptide-1), [H₂-Ala-His-Lys-OH]ₙ:copper(II), or [H₂-Ala-His-Lys-NH₂]ₙ:copper(II).

The symbol A1, A2, or the like used herein (e.g., in Formula 1) stands for the residue of an alpha-amino acid. Such symbols represent the general structure, —NH—CH(X)—CO— or ═N—CH(X)—CO— when it is at the N-terminus or —NH—CH(X)—CO— when it is not at the N-terminus, where X denotes the side chain (or identifying group) of the alpha-amino acid, e.g., X is —CH(CH₃)₂ for Val. Note that the N-terminus is at the left and the C-terminus at the right in accordance with the conventional representation of a polypeptide chain. R1 and R2 are both bound to the free nitrogen atom N-terminal amino acid (e.g., A1 or A2) and the R₃ is bound to the free carboxy group of the C-terminal amino acid (e.g., A3 or A4). Further, where the amino acid residue is optically active, it is the L-form configuration that is intended unless the D-form is expressly designated. An alkyl group, if not specified, contains 1-12 carbon atoms.

The amount of the copper peptide present in the composition will depend on the copper peptide used and the intended use of the composition. In one embodiment, the composition comprises a safe and effective amount of the copper peptide. The copper peptide typically will be present in the composition in an amount from about 0.001% to about 20% by weight, in particular in an amount from about 0.01% to about 1% by weight.

The method for synthesizing peptides of the present invention are well documented and are within the ability of a person of ordinary skill in the art. The synthesis of copper peptides of the present invention are set forth in U.S. Pat. Nos. 4,810,693 and 5,550,183.

Basic Amino Acid

What is meant by a “basic amino acid” is an amino acid that has a second basic group which may be an amino group such as lysine, a guanidino group such as argenine, or an imidazole ring such as histidine. Examples of basic amino acids include the D- and L-isomers of argenine, histidine, and lysine.

The amount of the basic amino acid present in the composition will depend on the basic amino acid used and the amount of copper peptide in the composition. In one embodiment, the composition comprises a safe and effective amount of the copper peptide. The basic amino acid typically will be present in the composition in an amount from about 0.001% to about 20% by weight, in particular in an amount from about 0.01% to about 5% by weight.

Compositions

The compositions useful in the present invention involve formulations suitable for topical application to skin. In one embodiment, the topical composition comprises the copper peptide and a cosmetically-acceptable topical carrier. In one embodiment, the cosmetically-acceptable topical carrier is from about 50% to abut 99.99%, by weight, of the composition (e.g., from about 80% to about 95%, by weight, of the composition.

The compositions may be made into a wide variety of product types that include but are not limited to lotions, creams, gels, sticks, sprays, shaving creams, ointments, cleansing liquid washes and solid bars, shampoos, pastes, powders, mousses, shaving creams, wipes, patches, nail lacquers, wound dressing and adhesive bandages, hydrogels, films and make-up such as foundations, mascaras, and lipsticks. These product types may comprise several types of cosmetically-acceptable topical carriers including, but not limited to solutions, emulsions (e.g., microemulsions and nanoemulsions), gels, solids and liposomes. The following are non-limitative examples of such topical carriers. Other topical carriers can be formulated by those of ordinary skill in the art.

The topical compositions useful in the present invention can be formulated as solutions. Solutions typically include an aqueous solvent (e.g., from about 50% to about 99.99% or from about 90% to about 99% of a cosmetically acceptable aqueous solvent).

Topical compositions useful in the subject invention may be formulated as a solution comprising an emollient. Such compositions preferably contain from about 2% to about 50% of an emollient(s). As used herein, “emollients” refer to materials used for the prevention or relief of dryness, as well as for the protection of the skin. A wide variety of suitable emollients are known and may be used herein.

A lotion can be made from such a solution. Lotions typically comprise from about 1% to about 20% (e.g., from about 5% to about 10%) of an emollient(s) and from about 50% to about 90% (e.g., from about 60% to about 80%) of water.

Another type of product that may be formulated from a solution is a cream. A cream typically comprises from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may comprise a simple base of animal or vegetable oils or semi-solid hydrocarbons. An ointment may comprise from about 2% to about 10% of an emollient(s) plus from about 0.1% to about 2% of a thickening agent(s).

The topical compositions useful in the present invention formulated as emulsions. If the carrier is an emulsion, from about 1% to about 10% (e.g., from about 2% to about 5%) of the carrier comprises an emulsifier(s). Emulsifiers may be nonionic, anionic, or cationic. However, it is preferred composition is substantially free of ionic emulsifiers (e.g., anionic or cationic emulsifiers). Examples of non-ionic emulsifiers include glyceryl stearate, PEG-100 stearate, cetearyl alcohol, cetearyl glucoside, steareths such as steareth-2, steareth-21, and steareth-25, ceteareths such as ceteareths-2 and ceteareth-100, ceteths such as ceteths-1 and ceteth-45, PEG-40 hydrogenated castor oil, lauryl polyglucose, dimethicone copolyol, polysiloxane polyalkyl/polyether copolymer, polymethacrylate polyacrylic/polyether copolymer, poloxamers(polymeric ethers), and polysorbates such as polsysorbate-20, polysorbate-60, and polysorbate-85.

Lotions and creams can be formulated as emulsions. Typically such lotions comprise from 0.5% to about 5% of an emulsifier(s). Such creams would typically comprise from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams,.of the oil-in-water type and water-in-oil type are well-known in the cosmetic art and are useful in the subject invention. Multiphase emulsion compositions, such as the water-in-oil-in-water type, are also useful in the subject invention. In general, such single or multiphase emulsions contain water, emollients, and emulsifiers as essential ingredients.

The topical compositions of this invention can also be formulated as a gel (e.g., an aqueous gel using a suitable gelling agent(s)) or contain a gelling agent. Suitable gelling agents for aqueous gels include, but are not limited to, gelatins, natural gums, polymers and copolymers of acrylic acids, acrylamides, carbamates, and/or acrylates, and cellulose derivatives (e.g., hydroxymethyl cellulose and hydroxypropyl cellulose). Examples of such include, but are not limited to, acrylamides copolymer, acrylamide/ammonium acrylate, acrylamides copolymer, acrylamide/sodium acrylate copolymer, carbomers, acrylates/acrylamide copolymers, and acrylates copolymer. Suitable gelling agents for oils (such as mineral oil) include, but are not limited to, hydrogenated butylene/ethylene/styrene copolymer and hydrogenated ethylene/propylene/styrene copolymer. Such gels typically comprises between about 0.1% and 5%, by weight, of such gelling agents.

The topical compositions of the present invention can also be formulated into a solid formulation (e.g., a wax-based stick, soap bar composition, powder, or a wipe containing powder).

Liposomal formulations are also useful compositions of the subject invention. Examples of liposomes are unilamellar, multilamellar, and paucilamellar liposomes, which may or may not contain phospholipids. The liposome preparation may then incorporated into one of the above carriers (e.g., a gel or an oil-in-water emulsion) in order to produce the liposomal formulation.

The topical compositions useful in the subject invention may contain, in addition to the aforementioned components, a wide variety of additional oil-soluble materials and/or water-soluble materials conventionally used in compositions for use on skin, hair, and nails at their art-established levels.

Additional Cosmetically Active Agents

In one embodiment, the topical composition further comprises another cosmetically active agent in addition to the copper peptide. What is meant by a “cosmetically active agent” is a compound that has a cosmetic or therapeutic effect on the skin, hair, or nails, e.g., lightening agents, darkening agents such as self-tanning agents, anti-acne agents, shine control agents, anti-microbial agents, anti-inflammatory agents, anti-mycotic agents, anti-parasite agents, external analgesics, sunscreens, photoprotectors, antioxidants, keratolytic agents, detergents/surfactants, moisturizers, nutrients vitamins, energy enhancers, anti-perspiration agents, astringents, deodorants, hair removers, firming agents, anti-callous agents, and agents for hair, nail, and/or skin conditioning.

In one embodiment, the cosmetically active agent is selected from, but not limited to, the group consisting of hydroxy acids, benzoyl peroxide, sulfur resorcinol, ascorbic acid, D-panthenol, hydroquinone, octyl methoxycinnimate, titanium dioxide, octyl salicylate, homosalate, avobenzone, polyphenolics, carotenoids, free radical scavengers, retinoids such as retinol and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, coenzyme Q10, lipoic acid, amino acids such a proline and tyrosine, vitamins, lactobionic acid, acetyl-coenzyme A, niacin, riboflavin, thiamin, ribose, electron transporters such as NADH and FADH2, and other botanical extracts such as aloe vera and soy, and derivatives and mixtures thereof.

The cosmetically active agent will typically be present in the composition of the invention in an amount of from about 0.001% to about 20% by weight of the composition, e.g., about 0.01% to about 10% such as about 0.1% to about 5%.

Examples of vitamins include, but-are not limited to, vitamin A, vitamin Bs such as vitamin B3, vitamin B5, and vitamin B12, vitamin C, vitamin K, and vitamin E and derivatives thereof.

Examples of hydroxy acids include, but are not limited, to glycolic acid, lactic acid, malic acid, salicylic acid, citric acid, and tartaric acid.

In one embodiment, the composition contains an antioxidant. Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopheryl acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, and propolis. Other examples of antioxidants may be found on pages 1612-13 of the ICI Handbook.

Other Materials

Various other cosmetically-active agents may also be present in the skin care products. These include, but are not limited to, skin protectants, humectants, and emollients. The skin care products may also comprise chelating agents (e.g., EDTA), preservatives (e.g., parabens), pigments, dyes, opacifiers (e.g., titanium dioxide), and fragrances. Preferably, the compositions are substantially free of formaldehyde.

Mineral Water

The compositions of the present invention may be prepared using a mineral water. In one embodiment, the mineral water has a mineralization of at least about 200 mg/L (e.g., from about 300 mg/L to about 1000 mg/L). In one embodiment, the mineral water comprises at least about 10 mg/L of calcium and/or at least about 5 mg/L of magnesium.

The composition and formulations containing such compositions of the present invention may be prepared using methodology that is well known by an artisan of ordinary skill.

### EXAMPLE 1

The following is an example of an oil-in-water emulsion of the present invention. The ingredients and their corresponding weight percentages are set forth in Table 1.

In the primary container the Carbomer and Xanthan Gum are dispersed in the Water and mixed until homogeneous. The Glycerin and Panthenol are then added and mixed in the primary container. The L-Argenine is then dissolved in water and added to the primary container. Following the gelling on the ingredients in the primary container, the primary container is then heated to 70° C.

The oil phase ingredients, namely C12-15 alkyl benzoate, Squalane, Dimethicone, Glyceryl Stearate (and) PEG-100 Stearate, Certeryl Alcohol (and) Cetearyl Glucoside, Soy Stearol, BHT, Tocopheryl Acetate, the parabens, and Phenoxyethanol are then combined and mixed in a second container and heated to 70° C. When both containers are at 70° C., the ingredients of the second container are then added to the primary container and allowed to mix until the emulsion is well formed. The resulting batch is then allowed to cool to 40-45° C. Copper Trieptide-1 is then dissolved in water and added and mixed in the batch. Lastly, the Aluminum Starch Octenylsuccinate is added to the batch. The batch is allowed to continue to mix as it cools to 30° C.

TABLE 1

| CTFA NAME | | WEIGHT % |
|---|---|---|
| WATER | | q.s. |
| CARBOMER | Synthalen M/ | 0.1%-3% |
| | 3V Chemical Corp. | |
| | Weehawken, NJ USA | |
| XANTHAN GUM | Keltrol CG/ | 0.1%-5% |
| | Calgon Corp | |
| | Pittsburgh, PA USA | |
| PANTHENOL | D Panthenol USP | 0.1%-5% |
| | BASF Corp. | |
| | North Mount Olive, | |
| | NJ, USA | |
| GLYCERIN | Pricerine/ | 0.1%-5% |
| | Uniqema | |
| | Wilmington, DE USA | |
| L-ARGENINE | Argenine/ | 0.1%-5% |
| | Ajinomoto | |
| | Teaneck, NJ USA | |
| C12-15 ALKYL | Finsolve TN/ | 0.1%-15% |
| BENZOATE | Fintex, Inc. | |
| | Elmwood Park, NJ | |
| | USA | |
| SQALANE | Phytosqualane/ | 0.1%-15% |
| | Sophim | |
| | Peyruis, France | |
| DIMETHICONE | Dow 200 | 0.1%-15% |
| | Fluid, 100 cst/ | |
| | Dow Corning | |
| | Midland, MI USA | |
| GLYCERYL STEARATE | Lipomulse 165/ | 0.1%-10% |
| (and) PEG-100 | Lipo Chemicals | |
| STEARATE | Inc. | |
| | Paterson, NJ USA | |
| CETEARYL ALCOHOL | Monthanov 86/ | 0.1%-10% |
| (and) CETARYL | Seppic Inc. | |
| GLUCOSIDE | Fairfield NJ 07006 | |
| SOY STEAROL | Generol 122 Cosm/ | 0.1%-5% |
| | Cognis Corp. | |
| | Ambler, PA USA | |
| BHT | Naugard BHT/ | 0.1%-1% |
| | Uniroyal Chem. | |
| | Hahnville, LA USA | |
| TOCOPHERYL ACETATE | Vitamin E Acetate | 0.1%-5% |
| | Hoffman La Roche | |
| | Parsippany, NJ USA | |
| ISPROPYLPARABEN | Liquapar Oil ISP/ | 0.1%-2% |
| (and) ISOBUTYL | Sutton Labs. | |
| PARABEN (and) BUTYL | Bound Brook, NJ | |
| PARABEN | USA | |
| PHENOXYETHANOL | Emeressence1160/ | 0.1%-2% |
| | Cognis Corp. | |
| | Ambler, PA USA | |
| ALUMINUM STARCH | Dry Flo-Pc/ | 0.1%-5% |
| OCTENYLSUCCINATE | National Starch & | |
| | Chemical | |
| | Monroe Township, | |
| | NJ USA | |
| COPPER TRIPEPTIDE-1 | Procyte | 0.001%-10% |
| | Corporation, | |
| | Redmond, WA USA | |

### EXAMPLE 2

The following is an example of an oil-in-water emulsions of the present invention. The ingredients and their corresponding weight percentages are set forth in Table 2.

Water is added to a first beaker. The Hydroxyethylcellulose is then added to the first beaker and mixed until dissolved. The Allantoin and L-Argenine are then added, and the resulting mixture is heated to 75-80° C. with moderate agitation.

In a second beaker, the following oil phase ingredients are added and mixed: Glyceryl Stearate, Stearic Acid, Cetyl Alcohol, Sodium Behenoyl Lactylate, PPG-2 Myristyl Ether Propionate, C12-15 Alkyl Benzoate, Tridecyl Stearate and Neopentyl Glycol Dicaprilate/Caprate and Tridecyl Trimelitate, BHT, Cyclopentasiloxane, Dimethicone, Tocopheryl Acetate, Avocado Oil Unsaponifiables, Squalane, Isopropylparaben, Isobutylparaben, and Butylparaben. These ingredients are mixed and heated to 75-80° C. until the waxes are completely melted.

While both beakers are maintained at 75-80° C., the mixture of the second beaker is added into to the mixture of the first beaker and mixed for 10 minutes until the emulsion was formed. The batch is then cooled to 40° C. and Diazolidiny Urea, Methylparaben, Propylparaben, and Propylene Glycol are added.

The Copper Tripeptide-1 is then dissolved in water and added to batch at 40° C. When the resulting mixture is completely mixed, the batch is then allowed to cool to room temperature.

TABLE 2

| CTFA NAME | TRADE NAME | WEIGHT % |
|---|---|---|
| WATER | | q.s. |
| L-ARGENINE | Argenine/ | 0.001-20% |
| | Ajinomoto | |
| | Teaneck, NJ USA | |
| GLYCERYL STEARATE | Lipo GMS 450/ | 0.1%-10% |
| | Lipo Chemicals Inc. | |
| | Paterson, NJ USA | |
| STEARIC ACID | Emersol 132/ | 0.1%-10% |
| | Henkel Corporation | |
| | La Grange, IL USA | |
| CETYL ALCOHOL | Cetal/ | 0.1%-10% |
| | Amerchol Corp. | |
| | Edison, NJ USA | |
| SODIUM BEHENOYL | Pationic SBL/ | 0.1%-10% |
| LACTYLATE | Rita Corporation | |
| | Woodstock, IL USA | |
| PPG-2 MYRISTYL ETHER | Crodamol PMP/ | 0.1%-10% |
| PROPIONATE | Croda, Inc. | |
| | Parsippany, NJ USA | |
| C12-15 ALKYL BENZOATE | Finsolve TN/ | 0.1%-20% |
| | Fintex, Inc. | |
| | Elmwood Park, | |
| | NJ USA | |
| BHT | Tenox BHT | 0.001-2% |
| | Eastman Chemical | |
| | Kingsport TN USA | |
| TRIDECYL STEARATE | Lipovol MOS-70/ | 0.1%-20% |
| (and) NEOPENTYL | Lipo Chemicals Inc. | |
| GLYCOL | Paterson, NJ USA | |
| DICAPRYLATE/CAPRATE | | |
| (and) TRIDECYL | | |
| TRIMELITATE | | |
| CYCLOPENTASILOXANE | DC 345/ | 0.1%-40% |
| | Dow Corning | |
| | Midland, MI USA | |
| DIMETHICONE | Dow 200 Fluid, 100 | 0.1%-10% |
| | cst/Dow Corning | |
| | Midland, MI USA | |
| DIAZOLIDINYL UREA | Germaben II E/ | 0.1%-2% |
| (and) METHYLPARABEN | International | |
| (and) PROPYLPARABEN | Specialties Products | |
| (and) PROPYLENE | Wayne, NJ USA | |
| GLYCOL | | |
| TOCOPHERYL ACETATE | Vitamin E Acetate/ | 0.1%-5% |
| | Hoffman La Roche, | |
| | Parsippany, NJ USA | |
| AVOCADO OIL | Avocadin/ | 0.1%-10% |
| UNSAPONIFIABLES | Croda Inc. | |
| | Parsippany NJ USA | |
| SQUALANE | (Phytosqualane/ | 0.1%-20% |
| | Sophim, | |
| | Parc de la Cassine, | |
| | 04310 Peyruis, France | |
| Copper Tripeptide-1 | Procyte Corporation, | 0.001%-10% |
| | Redmond, WA USA | |
| ISOPROPYLPARABEN & | Liquapar Oil | 0.1%-2% |
| ISOBUTYLPARABEN & | Sutton Labs. | |
| BUTYLPARABEN | Bound Brook, NJ USA | |
| HYDROXYETHYL | Cellosize HEC, | 0.001%-3% |
| CELLULOSE | PCG-10/Amerchol | |
| | Corporation Edison, | |
| | NJ USA | |
| ALLANTOIN | Sutton Labs | 0.001%-3% |
| | Bound Brook, NJ USA | |

### EXAMPLE 3

The following is an example of an oil-in-water emulsion of the present invention. The ingredients and their corresponding weight percentages are set forth in Table 3.

In the primary container, the Xanthan Gum was dispersed in the Water and mixed until homogeneous. The Glycerin, Polysorbate-20 (Unichema Wilmington, Del. USA), and Panthenol were then added and mixed in the primary container. The L-Argenine and Ultramarines (Warner & Jenkinson Company, Inc., St. Louis, Mo. USA) were then dissolved in water and added to the primary container. Following the gelling on the ingredients in the primary container, the Polyacrylamide (&) C13-14 Isoparaffin (&) Laureth-7 were then added to the primary container, which was then heated to 70° C.

The oil phase ingredients, namely Squalene, Dimethicone, Glyceryl Stearate (and) PEG-100 Stearate, Octinoxate (Interational Specialty Product, Wayne, N.J. USA), Octisalate (Haarman & Reimer, Rosemont, Ill. USA), Zinc Oxide (BASF, Washington, N.J. USA), Certeryl Alcohol (and) Cetearyl Glucoside, Soy Stearol, BHT, Tocopheryl Acetate, the parabens, and Phenoxyethanol were then combined and mixed in a second container and heated to 70° C. When both containers were at 70° C., the ingredients of the second container were then added to the primary container and allowed to mix until the emulsion was well formed. The resulting batch was then allowed to cool to 40-45° C. Copper Tripeptide-1 was then dissolved in water and added and mixed in the batch.

Lastly, the Aluminum Starch Octenylsuccinate was added to the batch. The batch was allowed to continue to mix as it cooled to 30° C.

TABLE 3

| CTFA NAME | WEIGHT % |
|---|---|
| WATER | 69.45 |
| XANTHAN GUM | 0.4 |
| PANTHENOL | 0.5 |
| GLYCERIN | 3.00 |
| L-ARGENINE | 0.03 |
| ULTRAMINES | 0.05 |
| POLYSORBATE-20 | 0.05 |
| OCTISALATE | 5.00 |
| OCTINOXATE | 7.50 |
| ZINC OXIDE | 2.00 |
| SQALANE | 0.50 |
| DIMETHICONE | 1.00 |
| GLYCERYL STEARATE (and) PEG-100 STEARATE | 2.00 |
| CETEARYL ALCOHOL (and) CETARYL GLUCOSIDE | 3.00 |
| SOY STEAROL | 0.25 |
| BHT | 0.07 |
| TOCOPHERYL ACETATE | 0.50 |
| ISPROPYLPARABEN (and) ISOBUTYL PARABEN (and) | 0.60 |
| BUTYL PARABEN | |
| PHENOXYETHANOL | 1.00 |
| POLYACRYLAMIDE (&) C13-14 | 1.00 |
| ISOPARAFFIN (&) LAURETH-7 | |
| ALUMINUM STARCH OCTENYLSUCCINATE | 2.00 |
| COPPER TRIPEPTIDE-1 | 0.15 |
| TOTAL | 100.00 |

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

1. A composition comprising:
(a) a peptide complexed with a copper ion; and
(b) a basic amino acid.

2. A composition of claim 1, wherein said peptide is of the formula 1:
R1[>A1-A2-His-A3-A4-R3]ₙ:copper(II) R2 Formula 1
wherein A1 is Gly or absent; A2 is Gly, Lys, Ala, Ser, or Val; A3 is Lys or Gly; A4 is Trp, (Gly)ₙ-Trp where n is from 1 to 4, Pro-Val-Phe-Val, Val-Phe-Val, or absent; each R1 and R2, independently, is H, C₁₋₁₂ alkyl, C₇₋₁₀ phenylalkyl, or C(═O)E₁, where E₁ is C₁₋₂₀ alkyl, C₃₋₂₀ alkenyl, C₃₋₂₀ alkynyl, phenyl, 3,4-dihydroxyphenylalkyl, naphthyl, or C₇₋₁₀ phenylalkyl; provided that when either R1 or R2 is C(═O)E₁, the other must be H; R3 is OH, NH2, C₁₋₁₂ alkoxy, C₇₋₁₀ phenylalkoxy, C₁₁₋₂₀ naphthylalkoxy, C₁₋₁₂ alkylamino, C₇₋₁₀ phenylalkylamino, or C₁₁₋₂₀ naphthylalkylamino; and n is 1 or 2.

3. A composition of claim 1, wherein said composition is an emulsion and further comprises a non-ionic emulsifier and said composition is substantially free of ionic emulsifiers.

4. A composition of claim 2, wherein said composition is an emulsion and further comprises a non-ionic emulsifier and said composition is substantially free of ionic emulsifiers.

5. A composition of claim 1, wherein said composition is an emulsion and further comprises an oil soluble anti-oxidant.

6. A composition of claim 5, wherein said composition is an emulsion and further comprises an oil soluble anti-oxidant.

7. A composition comprising:
(a) a peptide complexed with a copper ion; and
(b) a non-ionic emulsifier, wherein said composition is an emulsion and is substantially free of an ionic emulsifier.

8. A composition of claim 7, wherein said peptide is of the formula 1:
R1[>A1-A2-His-A3-A4-R3]ₙ:copper(II) R2 Formula 1
wherein A1 is Gly or absent; A2 is Gly, Lys, Ala, Ser, or Val; A3 is Lys or Gly; A4 is Trp, (Gly)ₙ-Trp where n is from 1 to 4, Pro-Val-Phe-Val, Val-Phe-Val, or absent; each R1 and R2, independently, is H, C₁₋₁₂ alkyl, C₇₋₁₀ phenylalkyl, or C(═O)E₁, where E₁ is C₁₋₂₀ alkyl, C₃₋₂₀ alkenyl, C₃₋₂₀ alkynyl, phenyl, 3,4-dihydroxyphenylalkyl, naphthyl, or C₇₋₁₀ phenylalkyl; provided that when either R1 or R2 is C(═O)E₁, the other must be H; R3 is OH, NH₂, C₁₋₁₂ alkoxy, C₇₋₁₀ phenylalkoxy, C₁₁₋₂₀ naphthylalkoxy, C₁₋₁₂ alkylamino, C₇₋₁₀ phenylalkylamino, or C₁₁₋₂₀ naphthylalkylamino; and n is 1 or 2.

9. A composition of claim 7, wherein said composition is an emulsion and further comprises an oil soluble anti-oxidant.

10. A composition of claim 8, wherein said composition is an emulsion and further comprises an oil soluble anti-oxidant.

11. A composition of claim 1, wherein said composition has a pH from about 6 to about 8.

12. A composition of claim 2, wherein said composition has a pH from about 6 to about 8.

13. A composition of claim 4, wherein said composition has a pH from about 6 to about 8.

14. A composition of claim 6, wherein said composition has a pH from about 6 to about 8.

15. A composition of claim 7, wherein said composition has a pH from about 6 to about 8.

16. A composition of claim 8, wherein said composition has a pH from about 6 to about 8.

17. A composition of claim 10, wherein said composition has a pH from about 6 to about 8.

18. A composition of claim 1, wherein said peptide is [H₂-Gly-L-His-L-Lys-OH]ₙ:copper(II) or [H₂-Gly-L-His-L-Lys-NH₂]ₙ:copper(II) and said basic amino acid is argenine, histidine, or lysine.

19. A composition of claim 4, wherein said peptide is [H₂-Gly-L-His-L-Lys-OH]ₙ:copper(II) or [H₂-Gly-L-His-L-Lys-NH₂]ₙ:copper(II) and said basic amino acid is aroenine, histidine, or lysine.

20. A composition of claim 6, wherein said peptide is [H₂-Gly-L-His-L-Lys-OH]ₙ:copper(II) or [H₂-Gly-L-His-L-Lys-NH₂]ₙ:copper(II) and said basic amino acid is argenine, histidine, or lysine.

21. A composition of claim 14, wherein said peptide is [H₂-Gly-L-His-L-Lys-OH]ₙ:copper(II) or [H₂-Gly-L-His-L-Lys-NH₂]ₙ:copper(II) and said basic amino acid is argenine, histidine, or lysine.

22. A composition of claim 7, wherein said peptide is [H₂-Gly-L-His-L-Lys-OH]ₙ:copper(II) or [H₂-Gly-L-His-L-Lys-NH₂]ₙ:copper(II).

23. A composition of claim 10, wherein said peptide is [H₂-Gly-L-His-L-Lys-OH]ₙ:copper(II) or [H₂-Gly-L-His-L-Lys-NH₂]ₙ:copper(II).

24. A composition of claim 17, wherein said peptide is [H₂-Gly-L-His-L-Lys-OH]ₙ:copper(II) or [H₂-Gly-L-His-L-Lys-NH₂]ₙ:copper(II).

25. A composition of claim 1, wherein said composition further comprises a gelling agent.

26. A composition of claim 7, wherein said composition further comprises a gelling agent.
